(19) United States
(12) Patent Application Publication
(10) Pub. No.: US 2017/0128379 A1
(43) Pub. Date: May 11, 2017

(54) MODIFIED RELEASE PHARMACEUTICAL PREPARATIONS

(71) Applicant: RUBICON RESEARCH PRIVATE LIMITED, Mumbai (IN)

(72) Inventors: Pratibha Sudhir PILGAONKAR, Mumbai (IN); Maharukh Tehmasp RUSTOMJEE, Mumbai (IN); Anilkumar Surendrakumar GANDHI, Mumbai (IN); Paras Rameshlal JAIN, Mumbai (IN)

(21) Appl. No.: 15/321,987

(22) PCT Filed: Jun. 30, 2015
(86) PCT No.: PCT/IN2015/000270

§ 371 Date: Dec. 23, 2016

(30) Foreign Application Priority Data
Jun. 30, 2014 (IN) 4144/MUM/2013

(51) Int. Cl. A61K 9/50; A61K 31/496; A61K 31/216; A61K 31/137; A61K 9/10

(52) U.S. Cl. CPC A61K 9/5047; A61K 31/137; A61K 9/10; A61K 9/5015; A61K 47/48184; A61K 31/216; A61K 31/496

(57) ABSTRACT

The present invention relates to modified release compositions comprising multitude of modified release beads. The modified release beads comprise a matrix of at least one active ingredient, at least one ion exchange resin, at least one non polymeric multifunctional excipient; substantially coated with at least one outer release rate modifying layer.

### FIELD OF THE INVENTION

The present invention relates to a modified release pharmaceutical preparation comprising multitude of modified release beads. Particularly the modified release beads comprise a matrix of drug-ion exchange resin complexes and at least one non-polymeric multifunctional excipient; substantially coated with at least one outer release rate modifying layer. The invention further relates to preparation of such modified release formulations in the form of liquid suspensions, tablets, capsules, orally disintegrating tablets, dispersible tablets, liquigels, beads, wafers, strips, and the like.

### BACKGROUND OF THE INVENTION

The modified release pharmaceutical formulations offer many advantages over conventional immediate release formulations such as modified blood levels, desired therapeutic effect for long period of time, attenuation of adverse effects, reduced side effects, reduction in the number of daily doses, improved patient convenience and compliance, etc.

Ion-exchange resin-drug complexes have been used to formulate improved pharmaceutical products of acidic and basic drugs. Ion exchange resins used in pharmaceutical applications serve a variety of functions, e.g., masking tastes, eliminating polymorphism, improving the dissolution of poorly soluble drugs, eliminating deliquescence, improving stability, reducing abuse liability and improving physical characteristics of pharmacologically active drugs. Ion exchange resins form a drug-resin complex by binding ionically to drugs. The drug molecules from the drug-resin complex are replaced by the external electrolyte present in normal physiological conditions in vivo or in a properly designed in vitro dissolution media, thereby releasing the bound drug over time. U.S. Pat. No. 2,990,332 discloses drug-resin complexes prepared by interaction of cationic ion-exchange resins with basic drugs in their cationic form, such as amphetamine and codeine. However, such uncoated complexes provide only a relatively short delay of drug release as well as poor control of drug release as they are affected by variation in particle size and cross-linkage in the resin, the ionic strength and pH of the gastrointestinal fluids and other conditions encountered in the gastrointestinal tract.

Therefore, attempts were made in certain instances to control the release rate of drug from the drug resin complex, to achieve the desired release rate and to provide the modified release for longer period of time, by providing a release modifier coating around discrete, minute, drug-ion exchange resin complex particles. The coating creates a diffusion barrier and thickness of which can be adjusted to provide the desired level of retardation of drug availability in the gastrointestinal tract over a period of time. Various coated drug-ion exchange resin complexes have been reported in U.S. Pat. Nos. 3,138,525, 3,499,960 and 3,594,470. One of the major disadvantage with the use of an ion exchange resin as a pharmaceutical delivery agent is that ion exchange resin drug complexes can undergo significant swelling when the dry, non-hydrated drug complex come in contact with fluids, e.g., water, biological fluids, gastrointestinal fluids. The swelling of the ion exchange resin often ruptures the diffusion barrier coating, which causes a loss of control of the diffusion rate of the drug, alters the drug release profile, and affects the dimensions and shape of the ion exchange resin-drug complex. Additionally, the coating can peel from the resin-drug complex and there can be instances of dose dumping. Also, due to swelling of drug-ion exchange resin complex and exchanging of drugs with ionic molecules from the physiological environment, the geometry of drug-resin complex is disrupted and the desired control over the rate of release of drug from drug resin complex is not achieved.

Some attempts were then made to control the swelling of the ion exchange resins to obtain desired controlled release. U.S. Pat. No. 4,221,778 teaches a pharmaceutical preparation employing drug-resin complex particles treated with a solvating agent and then coated with a diffusion barrier. The solvating or impregnating agent as discussed therein (e.g., polyethylene glycol, propylene glycol, mannitol, lactose and methylcellulose) retards the rate of swelling in water but does not reduce the overall amount of swelling, only the rate at which swelling occurs. U.S. Pat. No. 4,996,047 describes using drug content above a specified value in the drug-ion exchange resin complex to avoid the swelling of the drug-ion exchange resin complex, thereby minimizing the rupture of the coating and avoiding the need to use a solvating agent. U.S. Pat. No. 5,368,852 discloses that despite the use of impregnating agents, certain preservatives used in the liquid preparations containing coated drug-resin complexes tend to cause rupture of the diffusion barrier coating of the drug-ion exchange resin complex. Such a rupturing of the coating membrane is disclosed to be avoided first by use of a specific diffusion barrier film material, i.e. ethyl cellulose having a specific content of ethoxyl group, and secondly by adding the specific preservative at a specific concentration to control the release of the drug. US 2003/0099711 also describes use of ethyl cellulose polymer in an aqueous based coating system to control the swelling of drug-ion exchange resin complex and further use of enteric coating to delay the drug release. U.S. Pat. No. 8,062,667 describes admixing drug-ion exchange resin complex with a release retardant water insoluble polymer and coating such admixture with water insoluble, water permeable diffusion barrier coating.

Thus, researchers have developed drug-ion exchange resin based modified release formulations employing different techniques to control the swelling of the ion exchange resins and the ion exchange drug resin complexes. The disclosed formulations mainly employ polymeric substances to control swelling of drug-resin complex and the effectiveness of these materials is dependent on the complexity of the monomer and their internal crosslinking. Thus, most of the disclosed formulations and techniques of controlling the swelling of resins have drawbacks and are associated with a tendency to cause rupture of the release modifier coat and thereby leaching out of the drug and dose dumping.

A need therefore exists to provide modified release pharmaceutical compositions comprising modified release beads comprising drug resin complexes wherein the problem associated with swelling of the ionic resin is controlled, and the integrity of the release modifying coating is maintained, while preventing any dose dumping or high initial burst or excessive release retardation or incomplete release of the drug from the composition.

The present inventors after rigorous experimentation provide modified release preparations comprising multitude of modified release beads comprising a matrix of drug-ion exchange resin complexes and at least one multifunctional non-polymeric excipient, which is substantially coated with at least one outer release rate modifying layer. Without being bound to any theory, it is believed that the non-polymeric multifunctional excipient employed in the compositions of the present invention either alone or synergistically in combination with conventional solvating agent/s, help control any undesirable swelling of the ionic resin, maintain the geometry of drug-ion resin complex and prevent any rupture of the outer release modifying layer. Such a non-polymeric multifunctional excipient also provides cushioning effect around the drug-resin complexes and reduces the impact of swelling of the resins, if any, on the integrity of release modifier layer. Furthermore, the non-polymeric multifunctional excipient along with release modifier layer also provides dual control over the release of drug to achieve the desired rate of release of the active such as, but not limited to, once or twice a day formulations.

The modified release compositions according to the present invention achieve desired modified release profiles and do not cause either dose dumping or excessive release retardation and provide desired in-vitro drug release profile and bioavailability.

### SUMMARY OF THE INVENTION

The present invention relates to a modified release pharmaceutical preparation comprising multitude of modified release beads comprising pharmaceutically active agent, at least one ion exchange resin, at least one non-polymeric multifunctional excipient and substantially coated with at least one outer release rate modifying layer. Particularly the modified release beads comprise (a) a matrix of drug-ion exchange resin complexes and at least one non-polymeric multifunctional excipient; (b) substantially coated with at least one outer release rate modifying layer.

### DETAILED DESCRIPTION OF THE INVENTION

The present invention relates to modified release pharmaceutical formulation comprising multitude of modified release beads. Particularly the modified release beads comprise a matrix of drug-ion exchange resin complexes and at least one multifunctional excipient; substantially coated with at least one outer release rate modifying layer. The term “composition” or “formulation” or “dosage form” or “preparation” has been employed interchangeably for the purpose of the present invention and mean that it is a pharmaceutical formulation which is suitable for administration to a patient. For the purpose of the present invention, the terms “controlled release” or “sustained release” or “extended release” or “modified release” or “prolonged release” have been used interchangeably and mean broadly that the active agent is released at a predetermined rate that is different or slower than immediate release.

Active agents: The term “active agent/s”, as employed herein refers to any suitable drug that is capable of complexation with an ion exchange resin, and for which modified release is desired. In general all, including, but not limited to, acidic, basic, or amphoteric drugs, especially those having short biological half-lives such as in the order of up to about 12 hours are potential candidates in the compositions of the present invention. Active agents that exhibit a tendency to cause severe side effects when administered frequently in immediate release formulations are also potential candidates in the composition of the present invention. Active agents that can be included in the drug-resin complexes of the present invention include, but are not limited to, one or more psychostimulant, such as, but not limited to, amphetamine, amphetaminil, atomoxetine, dexmethylphenidate, dextroamphetamine, dextromethamphetamine, fencamfamine, fenethylline, lisdexamfetamine, methylphenidate, mesocarb, pemoline, pipradrol, prolintane and the like; or combinations thereof; antihistamines such as, but not limited to, dimenhydrinate, diphenhydramine, chlorpheniramine, brompheniramine, dexchlorpheniramine, hydroxyzine, dexbrompheniramine, fexofenadine, terfenadine, cetirizine, levocetirizine, fexofenadine hydrochloride or dl-chlorpheniramine maleate and the like; or combinations thereof; expectorants or mucolytics such as, but not limited to, ambroxol, bromhexine, carbocisteine, domiodol, guaifenesin and the like; or combinations thereof; anti-tussive agents such as, but not limited to, codeine, dextromethorphan, hydrocodone, dihydrocodeine phosphate, codeine phosphate, noscapine hydrochloride, phenylpropanolamine hydrochloride, potassium guaiacolsulfonate, cloperastine fendizoate, levocloperastine fendizoate, dextromethorphan hydrobromide, cloperastine hydrochloride; and the like; or combinations thereof; serotonin and norepinephrine reuptake inhibitor such as, but not limited to, clovoxamine, desvenlafaxine, duloxetine, levomilnacipran, eclanamine, milnacipran, sibutramine, venlafaxine, alaproclate, citalopram, escitalopram, femoxetine, fluoxetine, fluvoxamine, indalpine, ifoxetine litoxetine, omiloxetine, panuramine, paroxetine, pirandamine, seproxetine, sertraline zimelidine, and the like; or combinations thereof; sympatholytics such as, but not limited to, clonidine, guanfacine, methyldopa and the like; or combinations thereof; antipsychotics such as, but not limited to, iloperidone, ocaperidone, paliperidone, risperidone, lurasidone, perospirone, revospirone, tiospirone, ziprasidone, phenothiazine derivatives such as, but not limited to, chlorpromazine hydrochloride and the like or combinations thereof, and phenothiazine-like compounds, such as, but not limited to, chlorprothexene hydrochloride; and the like; or combinations thereof; antimuscarinics and urinary antispasmodics such as, but not limited to, darifenacin, emepronium, fesoterodine, flavoxate, imidafenacin, meladrazine, mirabegron, oxybutynin, propiverine, solifenacin, terodiline, tolterodine, trospium chloride and the like; or combinations thereof; PDE5 inhibitors such as, but not limited to, acetildenafil, aildenafil, avanafil, icariin, lodenafil, mirodenafil, nitrosoprodenafil, sildenafil, sulfoaildenafil, tadalafil, udenafil, vardenafil and the like; or combinations thereof; anti-Alzheimer's agent such as, but not limited to, memantine, neramexane (1, 3, 3, 5, 5-pentamethylcyclohexan-1-amine), donepezil, tacrine, rivastigmine, galantamine, physostigmine, neostigmine, Huperzine A, icopezil (CP-118954, 5,7-dihydro-3-[2-[1-(phenylmethyl)-4-piperidinyl]ethyl]-6H-pyrrolo-[4,5-f]-1,2-benzisoxazol-6-one maleate), ER-127528 (4-[(5,6-dimethoxy-2-fluoro-1-indanon)-2-yl]methyl-1-(3-fluorobenzyl) piperidine hydrochloride), zanapezil (TAK-147; 3-[1-(phenylmethyl)piperidin-4-yl]-1-(2, 3,4, 5-tetrahydro-1H-1-benzazepin-8-yl)-1-propane fumarate), metrifonate (T-588; (−)-R-α-[[2-(dimethylamino)ethoxy]methyl]benzo[b]thiophene-5-methanol hydrochloride), FK-960 (N-(4-acetyl-1-piperazinyl)-p-fluorobenzamide-hydrate), TCH-346 (N-methyl-N-2-pyropinyldibenz[b,f] oxepine-10-methanamine), SDZ-220-581 ((S)-α-amino-5-(phosphonomethyl)-[1,1′-biphenyl]-3-propionic acid), and the like or combinations thereof, tarenflurbil, tramiprosate, clioquinol and the like; or combinations thereof; analgesics such as, but not limited to, aspirin, codeine, morphine, dihydromorphine, oxycodone, hydrocodone, alfentanil, allyprodine, alphaprodine, anileridne, benzylmorphine, bezitramide, buprenorphine, butorphanol, clonitazene, cyclazocine, desmorphine, dextromoramide, dexocine, diampromide, dihydrocodeine, dimexoxadol, dimepheptanol, dimethylthiambutene, dioxaphetly butyrate, dipipanone, eptazocine, ethotheptazine, ethylmethylthiambutene, ethylmorphine, etonitazene fentanyl, hydromorphone, hydroxpethidine, isomethadone, ketobermidone, levallorphan, levorphanol, levophenacylmorphan, lofentanil, meperidine, meptazinol metazocine, methadone, metopon, morphine sulfate, myrophine, nalbuphine, narceine, cicomorphine, norlevorphanol, nomethadonel nalorphine, normophine, norpipanone, oxmymorphone, papaveretum, pentazocine, phenadoxone, phenmorphan, phenazocine, phenoperidine, iminodine, piritamide, propheptazine, promedol, properidine, propiram, proposyphene, sufenanil, tramadol, tiline and the like or combinations thereof; decongestants such as, but not limited to, phenylephrine, pseudoephedrine and the like or combinations thereof; analeptic agents; anesthetic agents; anti-asthmatics such as, but not limited to, theophylline and the like or combinations thereof; anti-arthritic agents; anti-cancer agents; anti-cholinergic agents; anti-convulsant agents such as, but not limited to, phenobarbital sodium, phenytoin sodium, valproate sodium barbiturates, amylobarbitone sodium, butabarbital sodium, secobarbital sodium and the like or combinations thereof; anti-depressant agents; antidiabetics; anti-helminthic agents; anti-diarrheal agents; anti-epileptics such as, but not limited to, phenytoin, meprobamate, nitrezepam and the like or combinations thereof; anti-hyperlipidemic agents; antihypertensives such as, but not limited to, clonidine, methyldopa; captopril, propranolol, hydralazine hydrochloride, propranolol hydrochloride, clonidine hydrochloride and the like or combinations thereof; antihypotensives, peripheral vasodilators/vasoconstrictors such as, but not limited to, tolazoline hydrochloride, respiradone, other respiratory agents such as, but not limited to, predinisolone, prednisolone sodium phosphate, albuterol, albuterol sulfate, terbutaline; and the like or combinations thereof; anti-infective agents; anti-inflammatory agents; non-steroidal anti-inflammatory agents such as, but not limited to, naproxen, diclofenac, indomethacin, ibuprofen, sulindac, meclofenamate sodium, tolmetin sodium and the like or combinations thereof; anti-emetics such as, but not limited to, metoclopramide and the like or combinations thereof; anti-migraine agents; anti-neoplastic agents; anti-tubercular agents; antibiotics such as, but not limited to, macrolides such as, but not limited to, oleandomycin phosphate and the like or combinations thereof, tetracyclines such as, but not limited to, tetracycline hydrochloride and the like or combinations thereof, streptomycins such as, but not limited to, fradiomycin sulfate, and the like or combinations thereof, and penicillin drugs such as, but not limited to, amoxicillin, dicloxacillin sodium, pivmecillinam hydrochloride, carbenicillin indanyl sodium, and the like or combinations thereof; and the like or combinations thereof; antacids; antiulcer agents; anti-Parkinsonism drugs such as, but not limited to, selegiline, rasagiline, entacapone, tolcapone; anti-pruritic agents; anti-pyretic agents; anti-spasmodics such as, but not limited, atropine, scopolamine, scopolamine hydrobromide, metixene hydrochloride, dicyclomine hydrochloride and the like or combinations thereof; anti-viral agents; appetite suppressants; attention deficit hyperactivity disorder treating agents; cardiovascular agents including, but not limited to, calcium channel blockers, antianginal agents; central nervous system agents; beta-blockers; antiarrhythmic agents; bronchodilators such as, but not limited to, albuterol, dl-methylephedrine hydrochloride and dl-methylephedrine saccharinate; central nervous system stimulants; diuretics such as, but not limited to, ethacrynic acid, bendrofluazide and the like or combinations thereof; genetic materials; hormonolytics; hypnotics; hypercalcemics; hypoglycemic agents; immunosuppressive agents; beta-agonists;

narcotic antagonists; nicotine; nutritional agents; parasympatholytics; peptide drugs; antihemorrhoidals; psychostimulants; psychotropics; mucolytics; sedatives; laxatives; vitamins; sialagogues, steroids; sympathomimetics; tranquilizers; vasodilators such as, but not limited to, nifedipine, papaverine, diltiazem, nicardipine, and the like; or combinations thereof; antianxiety drugs such as, but not limited to, benzodiazepine derivatives such as but not limited to, chlordiazepoxide hydrochloride, diazepam, and the like, alprazolam, and the like or combinations thereof, antidepressants such as, but not limited to, imipramine compounds such as but not limited to, imipramine hydrochloride, and the like, risperidone, SSRIs such as but not limited to, sertraline hydrochloride and the like, paroxitene hydrochloride, venlafaxine hydrochloride etc. and the like or combinations thereof; drugs for the treatment of respiratory system disorders such as, but not limited to, coronary dilators including, but not limited to, etafenone hydrochloride, calcium antagonists such as, but not limited to, verapamil hydrochloride and the like or any combinations thereof; antipyretic analgesics such as, but not limited to, sodium salicylate and the like or combinations thereof; hypnotics such as but not limited to phenobarbital sodium and the like or combinations thereof; chemotherapeutic drugs such as, but not limited to, sulfa drugs such as, but not limited to, sulfisomidine sodium, antituberculosis drugs such as, but not limited to, kanamycin sulfate, and antiprotozoan drugs such as, but not limited to, amodiaquine hydrochloride; and the like or combinations thereof. In one embodiment, combinations of one or more active agents listed above may also be employed in the compositions of the present invention. The active agent/s employed in the compositions of the present invention may be in the form of free base or acid or pharmaceutically acceptable salts, prodrugs, active metabolites, polymorphs, solvates, hydrates, enantiomers, optical isomers, tautomers or racemic mixtures thereof. In a further embodiment, acidic, basic or amphoteric drugs or combinations thereof may be delivered by the formulations of the present invention.

In another embodiment, one or more active agents such as, but not limited to, psychostimulants, antihistamines, expectorants or mucolytics, anti-tussive agents, serotonin and norepinephrine reuptake inhibitor, sympatholytics, antimuscarinics and urinary antispasmodics PDE5 inhibitors, anti-Alzheimer's agent, analgesics, decongestants, analeptic agents; anesthetic agents; anti-asthmatics, anti-arthritic agents; anti-cancer agents; anti-cholinergic agents; anti-convulsant agents, anti-depressant agents; antidiabetics; anti-helminthic agents; anti-diarrheal agents; anti-epileptics, anti-hyperlipidemic agents; antihypertensives, antihypotensives, peripheral vasodilators or vasoconstrictors, respiratory agents, anti-infective agents; anti-inflammatory agents; non-steroidal anti-inflammatory agents anti-emetics, anti-migraine agents; anti-neoplastic agents; anti-tubercular agents; antibiotics, antacids; antiulcer agents; anti-Parkinsonism drugs; anti-pruritic agents; antipsychotic agents; anti-pyretic agents; anti-spasmodics, anti-viral agents; anxiolytic agents; appetite suppressants; attention deficit hyperactivity disorder treating agents; cardiovascular agents, calcium channel blockers, antianginal agents; central nervous system agents; beta-blockers; antiarrhythmic agents; bronchodilators; central nervous system stimulants; diuretics; genetic materials; hormonolytics; hypnotics; hypercalcemics; hypoglycemic agents; immunosuppressive agents; beta-agonists; narcotic antagonists; nicotine; nutritional agents; parasympatholytics; peptide drugs; antihemorrhoidals; psychostimulants; psychotropics; mucolytics; sedatives; laxatives; vitamins; sialagogues, steroids; sympathomimetics; tranquilizers; vasodilators, antianxiety drugs, antidepressants, antipyretic analgesics, hypnotics, drugs for the treatment of respiratory system disorders, coronary dilators, calcium antagonists, chemotherapeutic drugs, antiprotozoan drugs and the like or combinations thereof may be employed. In a further embodiment, one or more active agents such as, but not limited to, amphetamine, amphetaminil, atomoxetine, dexmethylphenidate, dextroamphetamine, dextromethamphetamine, fencamfamine, fenethylline, lisdexamfetamine, methylphenidate, mesocarb, pemoline, pipradrol, prolintane, dimenhydrinate, diphenhydramine, chlorpheniramine, brompheniramine, dexchlorpheniramine, hydroxyzine, dexbrompheniramine, fexofenadine, terfenadine, cetirizine, levocetirizine, fexofenadine hydrochloride or dl-chlorpheniramine maleate, ambroxol, bromhexine, carbocisteine, domiodol, guaifenesin, codeine, dextromethorphan, hydrocodone, dihydrocodeine phosphate, codeine phosphate, noscapine hydrochloride, phenylpropanolamine hydrochloride, potassium guaiacolsulfonate, cloperastine fendizoate, levocloperastine fendizoate, dextromethorphan hydrobromide, cloperastine hydrochloride, clovoxamine, desvenlafaxine, duloxetine, levomilnacipran, eclanamine, milnacipran, sibutramine, venlafaxine, alaproclate, citalopram, escitalopram, femoxetine, fluoxetine, fluvoxamine, indalpine, ifoxetine litoxetine, omiloxetine, panuramine, paroxetine, pirandamine, seproxetine, sertraline zimelidine, clonidine, guanfacine, methyldopa, iloperidone, ocaperidone, paliperidone, risperidone, lurasidone, perospirone, revospirone, tiospirone, ziprasidone, chlorpromazine hydrochloride, chlorprothexene hydrochloride, darifenacin, emepronium, fesoterodine, flavoxate, imidafenacin, meladrazine, mirabegron, oxybutynin, propiverine, solifenacin, terodiline, tolterodine, trospium chloride, acetildenafil, aildenafil, avanafil, icariin, lodenafil, mirodenafil, nitrosoprodenafil, sildenafil, selegiline, rasagiline, entacapone, tolcapone, sulfoaildenafil, tadalafil, udenafil, vardenafil, memantine, neramexane (1, 3, 3, 5, 5-pentamethylcyclohexan-1-amine), donepezil, tacrine, rivastigmine, galantamine, physostigmine, neostigmine, Huperzine A, icopezil (CP-118954, 5,7-dihydro-3-[2-[1-(phenylmethyl)-4-piperidinyl]ethyl]-6H-pyrrolo-[4,5-f]-1,2-benzisoxazol-6-one maleate), ER-127528 (4-[(5,6-dimethoxy-2-fluoro-1-indanon)-2-yl]methyl-1-(3-fluorobenzyl) piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), metrifonate (T-588; (−)-R-α-[[2-(dimethylamino)ethoxy]methyl]benzo[b]thiophene-5-methanol hydrochloride), FK-960 (N-(4-acetyl-1-piperazinyl)-p-fluorobenzamide-hydrate), TCH-346 (N-methyl-N-2-pyropinyldibenz[b,f] oxepine-10-methanamine), SDZ-220-581 ((S)-α-amino-5-(phosphonomethyl)-[1,1′-biphenyl]-3-propionic acid), tarenflurbil, tramiprosate, clioquinol, aspirin, morphine, dihydromorphine, oxycodone, alfentanil, allyprodine, alphaprodine, anileridne, benzylmorphine, bezitramide, buprenorphine, butorphanol, clonitazene, cyclazocine, desmorphine, dextromoramide, dexocine, diampromide, dimexoxadol, dimepheptanol, dimethylthiambutene, dioxaphetly butyrate, dipipanone, eptazocine, ethotheptazine, ethylmethylthiambutene, ethylmorphine, etonitazene fentanyl, hydromorphone, hydroxpethidine, isomethadone, ketobermidone, levallorphan, levorphanol, levophenacylmorphan, lofentanil, meperidine, meptazinol metazocine, methadone, metopon, morphine sulfate, myrophine, nalbuphine, narceine, cicomorphine, norlevorphanol, nomethadonel nalorphine, normophine, norpipanone, ixmymorphone, papavretum, pentazocine, phenadoxone, phenmorphan, phenazocine, phenoperidine, iminodine, piritamide, propheptazine, promedol, properidine, propiram, proposyphene, sufenanil, tramadol, tiline, phenylephrine, pseudoephedrine, theophylline, phenobarbital sodium, phenytoin sodium, valproate sodium barbiturates, amylobarbitone sodium, butabarbital sodium, secobarbital sodium, phenytoin, meprobamate, nitrezepam, captopril, propranolol, hydralazine hydrochloride, propranolol hydrochloride, clonidine hydrochloride, tolazoline hydrochloride, predinisolone, prednisolone sodium phosphate, albuterol, albuterol sulfate, terbutaline, naproxen, diclofenac, indomethacin, ibuprofen, sulindac, meclofenamate sodium, tolmetin sodium, metoclopramide, oleandomycin phosphate, tetracycline hydrochloride, fradiomycin, sulfate, amoxicillin, dicloxacillin sodium, pivmecillinam hydrochloride, carbenicillin indanyl sodium, atropine, scopolamine, scopolamine hydrobromide, metixene hydrochloride, dicyclomine hydrochloride, dl-methylephedrine hydrochloride, dl-methylephedrine saccharinate; ethacrynic acid, bendrofluazide, nifedipine, papaverine, diltiazem, nicardipine, chlordiazepoxide hydrochloride, diazepam, alprazolam, imipramine hydrochloride, risperidone, sertraline hydrochloride, paroxitene hydrochloride, venlafaxine hydrochloride, sodium salicylate, etafenone hydrochloride, verapamil hydrochloride, sulfisomidine sodium, kanamycin sulfate, amodiaquine hydrochloride, dl-methyl-ephedrine hydrochloride, dehydrocholic acid, diflunisal, fenoprofen, furosemide, gemfibrozil, progencid, sulindac, salicylic acid, acetylsalicylic acid, acetophenazine, amitriptyline, benztropine, biperiden, bromodiphenhydramine, carbinoxamine, chloperastine, chlorcyclizine, chorpheniramine, chlorphenoxamine, chlorpromazine, clemastine, clomiphene, cyclizine, cyclobenzaprine, cyproheptadine, desipramine, dicyclomine, diphemanil, doxepin, doxylamine, ergotamine, fluphenazine, haloperidol, hydroxychloroquine, hyoscyamine, levopropoxyphene, maprotiline, meclizine, mepenzolate, meperidine, mephentermine, mesoridazine, metformin, methylepherdine, methdilazine, methscopolamine, methysergide, metoprolol, nortriptylene, noscapine, nylindrin, oxymorphone, orphenadrine, phendimetrazine, phentermine, phenylpropanolamine, pyrilamine, tripelennamine, triprolidine, promazine, propoxyphene, propanolol, quinidine, aminocaproic acid, aminosalicylic acid, isoxurprine, melphalan, nalidixic acid, paraaminosaliclic acid, chloropheniramine, niacin, methylphenidate hydrochloride, dexmethylphenidate hydrochloride, oxymorphone hydrochloride, hydrocodone bitartrate, albuterol sulfate, albuterol phosphate, chlorpheniramine maleate, metformin hydrochloride, oxybutynin hydrochloride, saligenine hydrochloride, cetrizine hydrochloride, ranitidine hydrochloride, and the like or combinations thereof in the form of free base or acid or pharmaceutically acceptable salts, prodrugs, active metabolites, polymorphs, solvates, hydrates, enantiomers, optical isomers, tautomers or racemic mixtures thereof may be employed in the compositions of the present invention. Pharmaceutically effective amount of active agent is employed in the composition of the present invention. The term “effective amount” refers to an amount effective to achieve desired preventive, therapeutic and/or beneficial effect. In one embodiment the amount of active agent in the composition can vary from about 0.01 weight % to about 85 weight %, based on the total weight of the composition. In another embodiment the amount of active agent in the composition can vary from about 0.02 weight % to about 75 weight %, based on the total weight of the composition. In still another embodiment, the amount of active agent in the composition can vary from about 0.05 weight % to about 60 weight %, based on the total weight of the composition.

Ion exchange resins: Active agent employed in the compositions of the present invention is complexed with at least one ion exchange resin. Ion exchange resins suitable for compositions of the present invention comprise a pharmacologically inert organic and/or inorganic matrix containing functional groups that are ionic or capable of being ionized under the appropriate conditions of pH. The organic matrix may be synthetic such as, but not limited to, polymers or copolymers of acrylic acid, methacrylic acid, sulfonated styrene, sulfonated divinylbenzene; or partially synthetic such as, but not limited to, modified cellulose and dextrans. The inorganic matrix includes, but is not limited to, silica gel modified by the addition of ionic groups. Covalently bound ionic groups may be strongly acidic (e.g., sulfonic acid, phosphoric acid), weakly acidic (e.g., carboxylic acid), strongly basic (e.g., primary amine), weakly basic (e.g. quaternary ammonium), or a combination of acidic and basic groups. The ion exchange resin having the polymeric matrix with an anionic functional group is a cation exchange resin and that having a cationic functional group is an anionic exchange resin. The mobile or exchangeable moieties depending on the type of resin can be but not limited to sodium, hydrogen, potassium, chloride and the like. In one embodiment of the present invention a cation exchange resin is employed for complexation with the active agent. Non limiting examples of suitable cation exchange resin that may be employed include Amberlite® IRP64 (porous copolymer of methacrylic acid and divinylbenzene), Amberlite® IRP69 (sodium polystyrene sulfonate or sulfonated copolymer of styrene and divinylbenzene), Amberlite® IRP88 (cross linked polymer of methacrylic acid and divinylbenzene), DOWEX® RTM. resins (strong cationic exchangers based upon polystyrene sulphonic acid with variable crosslinking (1-12% divinylbenzene)), Tulsion® 335—(Polacrilex/{Polacirilex S), Tulsion® 339 (Polacrilin potassium USP), Tulsion® 344 (Sodium polystyrene sulfonate BP), Indion® 204 (crosslinked polyacrylic acid), Indion® 214 (crosslinked polyacrylic acid), Indion® 234 (crosslinked polyacrylic acid), Indion® 234S (crosslinked polyacrylic acid), Indion® 294 (crosslinked polyacrylic acid), Purolite® C115 HMR (carboxylic acid functional group), Purolite® C115 E (carboxylic acid functional group), Purolite® C100 HMR (sulfonic acid functional group), Purolite® 100 MR (sulfonic acid functional group) or cation exchange resins having phosphonic functional groups. Cationic exchange resins are selected for use with basic active agents and molecules having a cationic functionality. Other suitable ion-exchange resins include anion exchange resins, such as have been described in the art and are commercially available. In one embodiment, the size of the ion-exchange particles that may be employed in the compositions of the present invention may be from about 5 microns to about 750 microns. In a further embodiment, the particle size is within the range of about 40 microns to about 250 microns for liquid dosage forms although particles up to about 1,000 micron can be used for solid dosage forms, e.g., tablets and capsules. In another embodiment, both regularly and irregularly shaped resin particles may be employed in the present invention. Regularly shaped particles are those particles that substantially conform to geometric shapes such as spherical, elliptical, and cylindrical and the like, which are exemplified by Dow XYS-40010.00 and Dow XYS-40013.00 (The Dow Chemical Company). Irregularly shaped particles are those particles that are not considered to be regularly shaped, such as particles with amorphous shapes and particles with increased surface areas due to surface channels or distortions. Irregularly shaped ion-exchange resins of this type are exemplified by Amberlite IRP-69 (Rohm and Haas). In one embodiment, the ion exchange resin used in the compositions of the present invention is sodium polystyrene sulfonate.

Drug-resin complexes: Drug-resin complexes according to the present invention comprise at least one active agent and at least one ion-exchange resin. In one embodiment basic active agent is complexed with cation exchange resin. In another embodiment active agent can be complexed with ion exchange resin in any ratio. In a further embodiment, ion exchange resin can be used for complexation with active agent in a ratio of active agent to resin of about 1:0.1 to about 1:20. In another embodiment, ion exchange resin can be used for complexation with active agent in a ratio of active agent to resin of about 1:0.25 to about 1:10. In still another embodiment, ion exchange resin can be used for complexation with active agent in a ratio of active agent to resin of about 1:0.5 to about 1:5. The terms “drug-ion exchange resin complexes” or “drug-resin complexes” have been used interchangeably for the purpose of the present invention. In one embodiment, the drug-ion exchange resin complexes can be prepared using methods known in the art, such as, but not limited to, blending, slurrying, kneading, grinding, sieving, filling, compressing, lyophilization, spray-drying, fluid-bed drying or centrifugal granulation. The drug-resin binding may be performed, for example, as a batch or column process, as is known in the art. In one illustrative embodiment, drug-resin complex is prepared by batch process. In one embodiment the drug-resin complexes were prepared by stirring aqueous slurry of drug and ion exchange resin for about 0.5 hours to about 12 hours, followed by filtration and drying of the formed drug-resin complex. In one embodiment, the invention relates to compositions comprising drug-resin complexes having one or more active agents. In another embodiment, the invention also relates to pharmaceutical compositions comprising drug-resin complexes wherein at least one pharmaceutically acceptable excipient has been employed during the process of preparation of the drug-resin complexes, such as, but not limited to, stabilizers and the like to inhibit or prevent degradation of the drug-resin complex during manufacturing process and over shelf life of the composition. Suitable stabilizers include, but are not limited to, antioxidants, chelating agents or combinations thereof. In one embodiment, stabilizer employed during the process of preparation of the drug-resin complexes is an antioxidant. Any suitable antioxidant agent available to those of ordinary skill in the art may be used. Antioxidant such as, but not limited to ascorbic acid, sodium metabisulphite, potassium metabisulfite, sodium bisulfite, sodium sulfite, tocopherol, sorbic acid, retinol, butylated hydroxyanisole (BHA), butylated hydroxytoluene (BHT), propyl gallate, sodium benzoate or any salt thereof, or a combination thereof may be employed. Any suitable chelating agent known to those of ordinary skill in the art may be used. Chelating agents such as, but not limited to, ethylene diaminetetraacetic acid (EDTA), desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid, bis(aminoethyl)glycolether-N,N,N′,N′-tetraacetic acid, iminodiacetic acid, citric acid, tartaric acid, fumaric acid, or any salt thereof, or a combination thereof may be employed. The stabilizing agent can be present in a concentration of about 0.001% to 20% by weight of the composition and may or may not be present in the final composition.

Non-polymeric multifunctional excipient: The drug-resin complexes along with at least one non-polymeric multifunctional excipient are present in the bead matrix. Without being bound to any theory, it is believed that the non-polymeric multifunctional excipient either alone or synergistically with the conventional impregnating or solvating agents help control the swelling of the drug-resin complex and protect the outer release modifying layer from any ruptures or disruptions that can occur due to the swelling of drug-resin complex by providing the cushioning effect around the drug-resin complex. The non-polymeric multifunctional agent also helps retain the geometry of drug-resin complex and enables the effective application of release modifying coatings to the drug-resin complexes thereby reducing surface cracking and retaining their ability to effectively prolong the release of drugs from drug resin complexes. Further, without being bound to any theory it is also believed that the non-polymeric multifunctional agent in combination with the release rate modifying layer provides dual control over release rate of drug for a desired period of time such as for, but not limited to once or twice a day administrations. The non-polymeric multifunctional agent helps avoid dose dumping and provides desired release rate of drug from the dosage form. In one embodiment, the non-polymeric multifunctional substance does not form a separate layer on the drug-ion resin complex but forms a matrix therewith. In another embodiment, the non-polymeric multifunctional substance is not applied in the form of a coating on the drug-ion resin complex. In a further embodiment, the non-polymeric multifunctional excipient is in the form of an admixture with the drug resin complexes. In a still further embodiment, the one or more non-polymeric multifunctional substances and the drug-resin complexes are in the form of granules. In another embodiment, the non-polymeric multifunctional substance and the drug-resin complexes are in the form of pellets.

In a further embodiment, the non-polymeric multifunctional excipient employed is a non-polymeric substance. In another embodiment, the non-polymeric multifunctional excipient employed includes, but is not limited to, fatty acid esters, waxes, fatty acids, fatty alcohols, hydrogenated vegetable oils and the like or any combinations thereof. In one embodiment, the fatty acid esters employed in the compositions of the present invention, include, but are not limited to, sucrose fatty acid esters, glyceryl fatty acid esters, fatty acid alkyl esters, propylene glycol esters of fatty acids, sorbitan fatty acid esters, or combinations thereof.

Sucrose fatty acid esters, generally called sugar esters are nonionic surfactants consisting of sucrose as hydrophilic group and fatty acid as lipophilic group. As sucrose has a total of 8 hydroxyl groups, compounds ranging from sucrose mono- to octa-fatty acid esters can be produced. Sucrose fatty acid esters that may be employed in the preparations of the present invention include, but are not limited to sucrose mono- to octa-fatty acid esters and various combinations and proportions thereof. Suitable sucrose fatty acid esters that may be employed include, but are not limited to, sucrose stearate, sucrose distearate, sucrose polystearate, sucrose palmitate, sucrose monopalmitate, sucrose dipalmitate, sucrose oleate, sucrose monooleate, sucrose dioleate, sucrose laurate, sucrose monolaurate, sucrose dilaurate, sucrose behenate, sucrose erucate, sucrose caprate, sucrose monocaprate, sucrose dicaprate, sucrose caprylate, sucrose monocaprylate, sucrose dicaprylate, sucrose myristate, sucrose monomyristate, sucrose dimyristate, sucrose linolenate, sucrose monolinolenate, sucrose dilinolenate, sucrose esters of mixed fatty acids, sucrose oligoesters, and mixtures thereof. In one embodiment, sucrose esters commercially available under the trademark SURFHOPE™ SE PHARMA from Mitsubishi Kagaku, Tokyo, or under the trademark SUCROESTER™ from Gattefosse and in many grades depending upon the fatty acid employed, the proportion of the mono- to octa-esters present and HLB value may be employed in the compositions of the present invention. In one embodiment, the sucrose fatty acid ester having HLB from about 1 to about 16 may be employed in the compositions of the present invention.

Glyceryl fatty acid esters that may be employed in the compositions of the present invention include, but are not limited to, mono-, di- and triglycerides. Suitable glyceryl fatty acid esters include, but are not limited to, glyceryl behenate; glyceryl monostearate, glyceryl tripalmitate, glyceryl monopalmitate, glyceryl distearate, glyceryl tristearate glyceryl palmitostearate, glyceryl dipalmitate, glyceryl monoleate, glyceryl monolaurate, glyceryl dilaurate, glyceryl trilaurate, glyceryl didocosanoate glycrl tridocosanoate, glyceryl monodocosanoate, glyceryl monocaprote, glyceryl dicaproate, glyceryl tricaproate, glyceryl monomyristate, giyberyl dimyistate, glyceryl trimyristate, glyceryl monodecenoate, glyceryl didecenoate, glycery! tridecenoate; GLYCOWAX-932; lauroyl macrogol-32 glycerides; stearoyl macrogol-32 glyceride; fatty acid esters such as those having a fatty acid chain length of about C₁₀-C.₄₀; glyceryl monoarachidonate, glyceryl monolinoleate, glyceryl monolinolenate, glyceryl monopalmitoleate, glyceryl monocaprylate, glyceryl monococoate, glyceryl monocollagenate, glyceryl monoerucate, glyceryl monohydroxystearate, glyceryl monoisopalmitate, glyceryl monolinoleate, glyceryl monolinolenate, glyceryl monomyristate, glyceryl monopentadecanoate, glyceryl monopolyacrylate, glyceryl monotallowate, glyceryl monothiopropionate, glyceryl monocundecylenate or mixtures thereof. Suitable fatty acid alkyl esters that may be employed in the compositions of the present invention include, but are not limited to, isopropyl monoarachidonate, isopropyl monolaurate, isopropyl monolinoleate, isopropyl monolinolenate, isopropyl monomyristate, isopropyl monopalmitoleate, isopropyl monooleate, and isopropyl monostearate; methyl monoarachidonate, methyl monolaurate, methyl monolinoleate, methyl monolinolenate, methyl monomyristate, methyl monopalmitoleate, methyl monooleate, methyl monostearate or mixtures thereof. Suitable propylene glycol esters of fatty acid that may be employed include, but are not limited to, propylene glycol monoarachidonate, propylene glycol monolaurate, propylene glycol monolinoleate, propylene glycol monolinolenate, propylene glycol monomyristate, propylene glycol monopalmitoleate, propylene glycol monooleate, propylene glycol monostearate or a combination thereof. Suitable sorbitan fatty acid esters that may be employed in the compositions of the present invention include, but are not limited to, sorbitan monolaurate, sorbitan monopalmitate, sorbitan monooleate, sorbitan monostearate, sorbitan tristearate, sorbitan trioleate, sorbitan sesquioleate, sorbitan tistearate, sorbitan monoisostearate, sorbitan sesquistearate, sorbitan caprylate, sorbitan cocoate, sorbitan diisostearate, sorbitan dioleate, sorbitan distearate, sorbitan isostearate, sorbitan olivate, sorbitan sesquiisostearate, sorbitan sesquistearate, and sorbitan triisostearate, and the like or combinations thereof.

In a further embodiment, suitable waxes may be employed in the compositions of the present invention. Waxes are esters of fatty acids with long chain monohydric alcohols. Natural waxes are often mixtures of such esters, and may also contain hydrocarbons. Waxes are low-melting organic mixtures or compounds having a high molecular weight and are solid at room temperature. Waxes may be hydrocarbons or esters of fatty acids and alcohols. Waxes employed in the present invention include, but are not limited to, natural waxes, such as animal waxes, vegetable waxes, and petroleum waxes (i.e., paraffin waxes, microcrystalline waxes, petrolatum waxes, mineral waxes), and synthetic waxes. Specific examples include, but are not limited to, spermaceti wax, carnauba wax, Japan wax, bayberry wax, flax wax, beeswax, Chinese wax, shellac wax, lanolin wax, sugarcane wax, candelilla wax, paraffin wax, microcrystalline wax, petrolatum wax, carbowax, and the like, or mixtures thereof. Mixtures of these waxes with the fatty acids may also be used. In another embodiment, suitable fatty acids may be employed in the compositions of the present invention. Fatty acids are carboxylic acids derived from or contained in an animal or vegetable fat or oil. Fatty acids are composed of a chain of alkyl groups containing from 4 to 22 carbon atoms and are characterized by a terminal carboxyl group. Fatty acids that may be employed in the present invention include, but are not limited to, decenoic acid, docosanoic acid, stearic acid, palmitic acid, lauric acid, myristic acid, and the like, and mixtures thereof. Suitable fatty alcohols that may be employed in the compositions of the present invention include, but are not limited to, cetyl alcohol, stearyl alcohol or mixtures thereof. Suitable hydrogenated vegetable oils that may be employed in the compositions of the present invention, include but are not limited to, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil, hydrogenated cottonseed oil, and the like, and mixtures thereof.

In one embodiment, the non-polymeric multifunctional excipient that may be employed in the pharmaceutical preparations of the present invention are sucrose fatty acid esters or mixtures thereof.

The amount of non-polymeric multifunctional substance used in the formulation may vary depending upon the medicament employed and the degree of modified release desired. In one embodiment, the non-polymeric multifunctional excipient present in the compositions of the present invention is in an amount from about 0.01% to about 90% by weight of the composition. In another embodiment, the non-polymeric multifunctional excipient present in the compositions of the present invention is in an amount from about 0.1% to about 85% by weight of the composition. In a further embodiment, the non-polymeric multifunctional excipient present in the compositions of the present invention is in an amount from about 0.5% to about 80% by weight of the composition.

Bead matrix: In one embodiment of the present invention, the drug-resin complexes and at least one non-polymeric multifunctional excipient form the matrix of the modified release beads or the bead matrix and this matrix is further substantially coated with the release rate modifying layer. In a further embodiment of the present invention, the matrix of modified release beads comprises drug-resin complexes and at least one non-polymeric multifunctional excipient. In a further embodiment, the matrix of modified release beads comprises drug-resin complexes, at least one non-polymeric multifunctional excipient and at least one pharmaceutically acceptable excipient discussed in the paragraphs beneath. In another embodiment, the matrix of modified release beads comprises drug-resin complexes, at least one non-polymeric multifunctional excipient, at least one impregnating agent or solvating agent and at least one pharmaceutically acceptable excipient discussed in the paragraphs beneath. In a further embodiment the various components of the bead matrix are present in various proportions in the modified release beads depending on the active used and the release profile desired. In another embodiment of the present invention, the matrix of modified release beads comprises drug-resin complexes in admixture with at least one non-polymeric multifunctional excipient. In a further embodiment, the matrix of the modified release beads comprises granules comprising drug-resin complexes and at least one non-polymeric multifunctional excipient In yet another embodiment, the matrix of the modified release beads comprises granules comprising drug-resin complexes, at least one multifunctional excipient, and at least one pharmaceutically acceptable excipient discussed in paragraphs beneath. In a further embodiment, the bead matrix is in the form of an admixture, granules, pellets and the like or mixtures thereof comprising drug-resin complexes and at least one non-polymeric multifunctional excipient. In one embodiment, the bead matrix is in the form of an admixture. In another embodiment, the bead matrix is in the form of granules. In a further embodiment, one or more different types of granules may be present in the bead matrix. In a further embodiment, one or more different types of granules that may be present in the bead matrix may comprise one or more different types of active agents, ion exchange resins, or non-polymeric multifunctional excipient. In a further embodiment, the matrix of the modified release beads comprises pellets comprising drug-resin complexes and at least one non-polymeric multifunctional excipient. In yet another embodiment, the matrix of the modified release beads comprises pellets comprising drug-resin complexes, at least one non-polymeric multifunctional excipient and at least one pharmaceutically acceptable excipient discussed in paragraphs beneath. In one embodiment, the bead matrix may be prepared by methods such as, but not limited to, physical mixing, wet granulation, dry granulation, melt granulation, and the like or any combinations thereof. In one embodiment, drug-resin complexes and at least one non-polymeric multifunctional excipient are dispersed uniformly in the bead matrix. In a further embodiment, drug-resin complexes and at least one non-polymeric multifunctional excipient are uniformly dispersed in bead matrix. In another embodiment, the non-polymeric multifunctional excipient may be added during the formation of the drug-resin complex or after the formation of the drug-resin complex or after drying of the drug-resin complexes or any combinations thereof.

Further, in one embodiment the drug-resin complexes and/or the bead matrix discussed above may be impregnated with any conventional solvating agent. In another embodiment, the drug-resin complexes and/or bead matrix may not be impregnated with any conventional solvating agent. In a further embodiment, the drug-resin complexes and/or bead matrix are impregnated with any conventional solvating agent. In one embodiment, the non-polymeric multifunctional agent may work synergistically with a conventional solvating agent. In one embodiment, the conventional solvating agent can be added as an ingredient in the resin drug complexation step or the drug resin complexes or the bead matrix can be treated with the conventional solvating agent after complexing. Solvating agent that can be employed in the compositions of the present invention include, but are not limited to, polyethylene glycol, propylene glycol, mannitol, lactose, methylcellulose, hydroxypropylmethylcellulose, sorbitol, polyvinylpyrrolidone, carboxypolymethylene, xanthan gum, propylene glycol alginate and combinations thereof. In an embodiment of the present invention along with any conventional solvating agent, at least one or more pharmaceutically acceptable excipients, such as but not limited to stabilizers may be employed for impregnation of the drug-resin complexes. The stabilizers that may be employed during impregnation of the drug-resin complexes include the ones as described above under drug-resin complexes.

Release rate modifying layer: In one embodiment, the bead matrix discussed above is substantially coated with a diffusion barrier coating of at least one release modifier. In another embodiment, the admixture of drug-resin complexes and at least one non-polymeric multifunctional agent are substantially coated with a diffusion barrier coating of at least one release modifier. In a further embodiment, the admixture of drug-resin complexes, at least one non-polymeric multifunctional agent and at least one pharmaceutically acceptable excipient are substantially coated with a diffusion barrier coating of at least one release modifier. In another embodiment, the admixture of impregnated or solvated drug-resin complexes, at least one non-polymeric multifunctional agent, and at least one pharmaceutically acceptable excipient are substantially coated with a diffusion barrier coating of at least one release modifier. In a further embodiment, the solvated bead matrix comprising an admixture of drug resin complexes and non-polymeric multifunctional excipient is substantially coated with a diffusion barrier coating of at least one release modifier. In another embodiment, the solvated bead matrix comprising an admixture of solvated drug-resin complexes and non-polymeric multifunctional excipient is coated with a diffusion barrier coating of at least one release modifier.

In a further embodiment, the granules of drug-resin complexes and at least one non-polymeric multifunctional agent are substantially coated with a diffusion barrier coating of at least one release modifier. In a further embodiment, the granules of drug-resin complexes, at least one non-polymeric multifunctional agent and at least one pharmaceutically acceptable excipient are substantially coated with a diffusion barrier coating of at least one release modifier. In one embodiment, the granules of drug-resin complexes, at least one non-polymeric multifunctional agent and optionally at least one pharmaceutically acceptable excipient and optionally admixture of drug-resin complexes, at least one non-polymeric multifunctional agent and optionally at least one pharmaceutically acceptable excipient are substantially coated with at least one release modifier. In a further embodiment the granules of drug-resin complexes, at least one non-polymeric multifunctional agent and optionally at least one pharmaceutically acceptable excipient in admixture with at least one pharmaceutically acceptable excipient may be substantially coated with at least one release modifier coating. In another embodiment, the granules of impregnated or solvated drug-resin complexes, at least one non-polymeric multifunctional agent, and at least one pharmaceutically acceptable excipient are substantially coated with a diffusion barrier coating of at least one release modifier. In a further embodiment, the solvated bead matrix comprising granules of drug resin complexes and non-polymeric multifunctional excipient are substantially coated with a diffusion barrier coating of at least one release modifier. In another embodiment, the solvated bead matrix comprising granules of solvated drug-resin complexes and non-polymeric multifunctional excipient is coated with a diffusion barrier coating of at least one release modifier. In a further embodiment, bead matrix in any form may be substantially coated with at least one release modifier.

The terms “release modifier” or “release rate modifier” have been used interchangeably for the purpose of the present invention and refer to a substance or a combination of substances that can help modify the release of active agent from the formulation. The release modifiers that may be employed in the compositions of the present invention include, but are not limited to, water-insoluble release modifiers or water-soluble release modifiers or combinations thereof. The water-insoluble release modifiers that may be employed include polymeric water-insoluble release modifier or non-polymeric water-insoluble release modifier or combinations thereof. Suitable polymeric water-insoluble release modifiers include, but are not limited to, cellulose polymers and derivatives thereof, polyacrylic acid and polymethacrylic acid polymers and derivatives thereof, maleic acid copolymers and derivatives thereof, polyvinyl derivatives; and the like or any combinations thereof. In one embodiment, suitable polymeric water-insoluble release modifiers include, but are not limited to, polyvinyl acetate, polyvinyl chloride, polyvinyl carbonate, ethyl cellulose, nitrocellulose, vinylidene chloride-acrylonitrile copolymer, acrylonitrile-styrene copolymer, ethylene vinyl acetate, cellulose acetate, cellulose acetate phthalate, cellulose acetate butyrate, copolymers of vinyl pyrrolidone, blend of polymers comprising polylvinyl acetate, hydroxypropylmethylcellulose phthalate, methacrylic acid copolymers such as Eudragit® LI00/SI00/LI00-55 and the like or mixtures thereof; methacrylate copolymers such as Eudragit® E100/EPO, Eudragit® RL100/RL30D/RLPO, Eudragit® RS100/RS30D/RSPO and the like or mixtures thereof. Suitable non-polymeric water-insoluble release modifiers include, but are not limited to, fats, oils, waxes, fatty acids, fatty acid esters, glycerides, long chain monohydric alcohols and their esters, phospholipids, terpenes or combinations thereof. Suitable release modifiers in each of these categories have been listed hereinbefore. The non-polymeric water-insoluble release modifiers that may be employed in the compositions of the present invention include, but are not limited to, Cutina® (hydrogenated castor oil), Hydrobase® (hydrogenated soybean oil), Castorwax® (hydrogenated castor oil), Croduret® (hydrogenated castor oil), Carbowax®, Compritol® (glyceryl behenate), Sterotex® (hydrogenated cottonseed oil), Lubritab® (hydrogenated cottonseed oil), Apifil® (wax yellow), Akofine® (hydrogenated cottonseed oil), Softisan® (hydrogenated palm oil), Hydrocote® (hydrogenated soybean oil), Corona® (Lanolin), Gelucire® (macrogolglycerides Lauriques), Precirol® (glyceryl palmitostearate), Emulcire™ (cetyl alcohol), Plurol® diisostearique (polyglyceryl diisostearate), Geleol® (glyceryl stearate), and mixtures thereof. In another embodiment, lipids or waxes can also be employed in the form of an aqueous dispersion stabilized by surfactants and suitable stabilizers. Suitable water soluble release modifiers that may be employed include, but are not limited to, cellulose polymers and derivatives thereof, gums, polyvinyl derivatives and the like or combinations thereof. In one embodiment, suitable water soluble release modifiers that may be employed include, but are not limited to, polyvinylpyrrolidone, poloxamer, guar gum, xanthan gum, fenugreek gum or galactomannan, gum arabic, fenugreek fibers comprising soluble and insoluble fibers, tragacanth, cellulose derivatives such as hydroxypropylmethylcellulose, hydroxypropyl cellulose, methylcellulose, and hydroxyethyl cellulose, carboxymethylethyl cellulose, hydroxyethylmethyl carboxymethyl cellulose, hydroxyethyl methyl cellulose, carboxymethyl cellulose, methylhydroxyethyl cellulose, methylhydroxypropyl cellulose or any mixtures thereof. In one embodiment the release modifier employed is ethyl cellulose. The release modifiers of the present invention may be used in admixture with at least one pharmaceutically acceptable excipient, such as but not limited to, plasticizers, pigments, anti-tacking agents and the like or any mixtures thereof. Suitable plasticizers include, but are not limited to, dibutyl sebacate, propylene glycol, polyethylene glycol, polyvinyl alcohol, triethyl citrate, acetyl triethyl citrate, acetyl tributyl citrate, tributyl citrate, triacetin or the like or any combinations thereof. Suitable anti-tacking agents that may be employed include, but are not limited to, talc, colloidal silicon dioxide and the like or combinations thereof. In a further embodiment, stabilizers as described under drug-resin complexes may be employed in the release rate modifier layer.

A coating procedure known to a person skilled in the art, which provides a substantially complete coating on the bead matrix, without significant agglomeration of the drug-resin complex particles, may be used. In a further embodiment, the bead matrix in the form of an admixture, granules, pellets and the like or mixtures thereof comprising drug-resin complexes and at least one non-polymeric multifunctional excipient, may be coated by a process known to a person skilled in the art, without significant agglomeration of the drug-resin complex particles. Coating may be applied to the bead matrix by processes such as, but not limited to, melt coating, spray coating, pan coating, fluidized bed coating and the like. Coatings may be applied in a coating pan or with a fluid-bed coating apparatus. The release modifier coatings may be applied from aqueous suspension or organic solvents such as, but not limited to, isopropyl alcohol. Optionally after coating the coated drug-resin complexes may be cured at a suitable temperature and for a suitable amount of time. The term “substantially coated” as used herein means that the bead matrix in the forms discussed herein above is substantially completely coated with the release modifier. While complete coating over the bead matrix particles, with release modifier is ideal, minor variations in this are possible in practice during coating and are therefore referred to as “substantially coated”.

In one embodiment, modified release beads comprising only coated modified release beads are incorporated in the compositions of the present invention. Optimum coat weight and coat thickness may be determined and generally depends on the drug release characteristics of the resin for that particular active agent. In one embodiment, the bead matrix may be coated with at least one release modifier to a weight gain of about 1% to about 75%. In a further embodiment, the bead matrix is coated to a weight gain of about 1% to about 70%. In one embodiment the bead matrix is variably coated at different levels of release modifier coating and the variably coated beads are present in particular proportions in the modified release compositions. The presence of such variably coated beads helps achieve the desired release profiles that does not result either in dose dumping or excessive release retardation. In one embodiment the compositions of the present invention comprise at least two variably coated populations or portions of coated beads or the coated beads are present in the form of at least two populations of variably coated bead matrices. In another embodiment at least two populations of variably coated drug-resin complexes are present in a ratio from about 1:99 to about 99:1. In one embodiment, coated and uncoated modified release beads may be incorporated in the compositions of the present invention.

Modified release beads: In one embodiment the coated bead matrices may be present in the modified release beads of the present invention in an amount of from about 10% to about 100% by weight of the beads. In a further embodiment the coated bead matrices may be present in the modified release beads of the present invention in an amount of from about 20% to about 100% by weight of the beads. In another embodiment the modified release beads comprise coated bead matrices and at least one pharmaceutically acceptable excipient such as, but not limited to, diluents, stabilizers, or the like as discussed hereinafter or any combinations thereof. In one embodiment the modified release beads of the present invention comprise one or more populations of bead matrices coated at different levels with the release modifier coating. In a further embodiment, the compositions of the present invention comprise one or more populations of modified release beads. The modified release beads employed in the present invention are in forms such as, but not limited to, particles, granules, pellets, beads, minitablets, tablets and the like or any combinations thereof. The modified release beads may be present in the compositions in an amount from about 5% to about 95% by weight of the composition. The modified release beads are incorporated in the modified release pharmaceutical formulations of the present invention by any of the methods generally known to a person skilled in the art, especially depending on the form of the modified release beads incorporated and the final form of the pharmaceutical composition.

Modified release formulations: Modified release pharmaceutical compositions of the present invention comprise multitude of modified release beads and at least one pharmaceutically acceptable excipient. The modified release compositions may be formulated for delivery of active agent by any suitable route including, e.g. orally, topically, intraperitoneally, transdermally, sublingually, intramuscularly, transmucosally, rectally, subcutanoeulsly, transnasally or via inhalation. In one embodiment, the modified release compositions are for oral delivery. The compositions for oral delivery may be in any form, such as, but not limited to, liquid, solid or semi-solid preparations and the like. Liquid preparations for oral administration may be in any form including, but not limited to, suspensions, syrups or the like. Solid preparations for oral administration may be in any form including, but not limited to, capsules, tablets, caplets, orally disintegrating tablets, dispersible tablets, dry suspension for reconstitution, granules, wafers, bite-dispersion tablets and the like or any combinations thereof. In one embodiment the modified release preparation of the present invention is a suspension. The modified release compositions of the present invention comprise at least one pharmaceutically acceptable excipient, depending on the final dosage form to be prepared, such as, but not limited to, binders, disintegrants, superdisintegrants, diluents, salivating agents, surfactants, flavors, sweeteners, colorants, souring agents, viscolizers, glidants, chelating agents, lubricants, solubilizers, stabilizers, suspending agents, preservatives, cosolvents, anti-caking agents, buffers and the like or any combinations thereof. Suitable disintegrants can be selected from, but not limiting to, crospovidone, calcium silicate and starch. Suitable superdisintegrants include, but are not limited to, natural, modified or pregelatinized starch, crospovidone, croscarmellose sodium, sodium starch glycolate, low-substituted hydroxypropyl cellulose. Examples of suitable binders include, but are not limited to, starch, pregelatinized starch, polyvinyl pyrrolidone, copovidone, cellulose derivatives, such as hydroxypropylmethyl cellulose, hydroxypropyl cellulose and carboxymethyl cellulose and their salts. Examples of suitable diluents include, but are not limited to, starch, microcrystalline cellulose, lactose, xylitol, mannitol, maltose, polyols, fructose, guar gum, sorbitol, magnesium hydroxide, dicalcium phosphate, coprocessed mannitol and calcium silicate and the like or any combinations thereof. Examples of lubricants include, but are not limited to, magnesium stearate, calcium stearate, stearic acid, talc, and sodium stearyl fumarate. Suitable glidants includes but are not limited to, colloidal silica, silica gel, precipitated silica, or combinations thereof. Suitable salivating agents include, but are not limited to, micronised polyethylene glycol, sodium chloride or precipitated micronised silica. Examples of solubilizers include, but are not limited to cetostearyl alcohol, cholesterol, diethanolamine, ethyl oleate, ethylene glycol palmitostearate, glycerin, glyceryl monostearate, isopropyl myristate, lecithin, medium-chain glyceride, monoethanolamine, oleic acid, propylene glycol, polyoxyethylene alkyl ether, polyoxyethylene castor oil glycoside, polyoxyethylene sorbitan fatty acid ester, polyethylene sorbitan fatty acid ester, polyoxyethylene stearate, propylene glycol alginate, sorbitan fatty acid ester, stearic acid, sunflower oil, triethanolmine, or combinations thereof. Souring agents include, but are not limited to, monosodium fumarate and/or citric acid. The compositions of the present invention may also include stabilizers such as, but not limited to, those described above under drug-resin complexes. Suitable chelating agents that may be employed have been discussed herein above. Suitable viscolizers include, but are not limited to, coprocessed microcrystalline cellulose such as but not limited to, Avicel RC591, Avicel CL-611, D-sorbitol solution, polyalkylene oxides such as, but not limited to polyethylene oxide; cellulose ethers such as, but not limited to hydroxyethyl cellulose, hydroxypropylcellulose, hydroxypropyl methyl cellulose, methyl cellulose, ethyl cellulose, sodium carboxy methylcellulose, calcium carboxymethyl cellulose, microcrystalline cellulose; gums such as but not limited to gum arabic alginates, agar, sodium alginate guar gum, locust bean, carrageenan, tara, gum arabic, tragacanth, pectin, xanthan, gellan, maltodextrin, galactomannan, pusstulan, laminarin, scleroglucan, gum arabic, inulin, karaya, whelan; polyols such as, but not limited to dipropylene glycol, polypropylene glycol, propylene glycol, polyethylene glycol (PEG), sorbitol and glycerol; carbopol, starch and starch-based polymers such as, but not limited to, pregelatinized starch, acrylic acid and methacrylic acid polymers, and esters thereof, maleic anhydride polymers; polymaleic acid; poly(acrylamides); poly(olefinic alcohol)s; poly(N-vinyl lactams); polyoxyethylated saccharides; polyoxazolines; polyvinylamines; polyvinylacetates; polyimines; povidone, vinylpyrrolidone/vinyl acetate copolymer and polyvinyl acetate, mixture of polyvinyl acetate and polyvinylpyrrolidone, chitin, cyclodextrin, gelatin, chitosan and the like or any mixtures thereof. Suitable surfactants include, but are not limited to, anionic, nonionic, cationic, and zwitterionic surfactants or a mixture thereof. The non-ionic surfactants employed in the composition may include, but are not limited to, ethoxylated fatty acid ester, ethoxylated fatty acid ethers, ethoxylated sorbitan ethers, ethoxylated alkyl-phenols, glycerol esters, glycerol sugar esters, polyoxyethyleneglycerol monolaurate, polyoxyethyleneglycerol monostearate, polyoxyethylene-20-cetyl stearate, polyoxyethylene-25-cetyl stearate, polyoxyethylene (25)-oxypropylene monostearate, polyoxyethylene-20-sorbitan monopalmitate, poly-oxyethylene-16-tert-octylphenol, polyoxyethylene-20-cetyl ether, polyethylene glycol(1000) monocetyl ether, ethoxylated castor oil, polyoxyethylene sorbitol-lanolin derivatives, polyoxyethylene(25)propylene glycol stearate, polyoxyethylenesorbitol esters, polyoxyethylene-20-sorbitan monopalmitate, polyoxyethylene-16-tert-octylphenol, polyoxyethylene-20-cetyl ether, glycyeryl undecylenate and Polysorbate 60, capmul (medium chain glyceride), peceol (glyceryl monooleate), glyceryl laurate and glyceryl caprylate (Capmul MCM), PEG sorbitan fatty acid esters like PEG-20 sorbitan monolaurate (Tween 20), PEG-20 sorbitan monostearate (Tween 60), PEG-20 sorbitan monooleate (Tween 80), sorbitan fatty acid esters like sorbitan monolaurate (Span 20), glyceryl stearate (Cithrol GMS) or the like and mixtures thereof. Suitable cationic surfactants include, but are not limited to, quaternary ammonium compounds, alkylamidoamines and quaternary ester compounds, distearyl dimethyl ammonium chloride, dimyristyl dimethyl ammonium chloride, dipalmityl dimethyl ammonium chloride or the like and mixtures thereof. Suitable anionic surfactants include, but are not limited to, fatty alcohol sulfates, alpha olefin sulfonates, sulfosuccinates, phosphate esters, carboxylates, sarcosinates, alkyl benzene sulfonates, alkyl sulfonates, olefin sulfonates, alkyl ethersulfonates, glycerol ethersulfonates, α-methyl estersulfonates, sulfonic fatty acids, alkyl sulfates, fatty alcohol ethersulfates, glycerol ethersulfates, mixed hydroxy ethersulfates, monoglyceride (ether)sulfates, fatty acid amide (ether)sulfates, sulfosuccinates, sulfosuccinamates, sulfotriglycerides, amide soaps, ether carboxylic acids, isethionates, sarcosinates, taurides, alkyl oligoglycoside sulfates, alkyl (ether)phosphates or the like and mixtures thereof. Suitable zwitterionic surfactants employed include, but are not limited to, N-alkyl-N,N-dimethyl ammonium glycinates, for example cocoalkyl dimethyl ammonium glycinate, N-acyl aminopropyl-N,N-dimethyl ammonium glycinates, cocoacyl aminoethyl hydroxyethyl carboxymethyl glycinate or the like and mixtures thereof. Further, the composition of the present invention may further comprise a preservative such as but not limited to methyl parahydroxybenzoate, propyl parahydroxybenzoate and sodium benzoate. Suitable cosolvent that may be used includes, but is not limited to, ethanol and polyhydric alcohols such as, but not limited to, glycerin, propylene glycol, low molecular weight polyethylene glycols, and mixtures thereof. Further anti-caking agents that may be optionally incorporated include, but are not limited to, colloidal silicon dioxide, tribasic calcium phosphate, powdered cellulose, magnesium trisilicate, starch, and mixtures thereof. Suitable sweetening agent includes, but is not limited to, aspartame, stevia extract, glycyrrhiza, saccharine, saccharine sodium, acesulfame, sucralose, dipotassium glycyrrhizinate, galactose, fructose, high fructose corn syrup, dextrose, sucrose, sugar, maltose, partially hydrolyzed starch, corn syrup solids, sorbitol, xylitol, mannitol and the like or mixtures thereof. The compositions may comprise one or more natural and/or artificial flavors such as, but not limited to, mint flavour, orange flavour, lemon flavors, strawberry aroma, vanilla flavour, raspberry aroma, cherry flavor, tutty frutty flavor, magnasweet 135, key lime flavor, grape flavor, trusil art 511815, and fruit extracts and the like. Suitable colorants include, but are not limited to, pigments and dyes such as FD&C Red, FD&C Yellow, FD&C Green, and FD&C Blue and the like or combinations thereof. In one of the embodiment, the solid dosage form of the present invention may be optionally coated. Surface coating may be employed for aesthetic purposes or for dimensionally stabilizing the compressed dosage form. The coating may be carried out using any conventional technique employing conventional ingredients suitable for oral use. A surface coating can, for example, be in the form of a film using conventional polymers including, but not limited to, hydroxypropyl methyl cellulose, hydroxypropyl cellulose, carboxymethyl cellulose, polyvinyl alcohol polymethacrylates and the like, and combinations thereof. In another embodiment of the present invention, the composition may be optionally coated with a functional coat. The functional coat may be applied using coating agents including, but not limited to, hydrophilic polymers, hydrophobic polymers, waxes, and the like, or mixtures thereof, either alone or in combination, along with plasticizers, colorants, opacifiers etc. The functional coat may help provide the desired drug release profile.

The modified release compositions of the present invention can be readily formulated according to methods well known to those skilled in the art. Method of preparation of the compositions of the present invention depends on the final dosage form desired. In one embodiment, the composition of the present invention is in the form of a modified release suspension. In another embodiment, the modified release suspension of the present invention comprises multitude of modified release beads comprising a matrix of drug-ion exchange resin complex and at least one sucrose fatty acid ester; substantially coated with at least one outer release rate modifying layer. In a further embodiment, the modified release suspension of the present invention comprises multitude of modified release beads comprising a matrix of drug-ion exchange resin complex and at least one sucrose fatty acid ester; substantially coated with ethyl cellulose.

The compositions of the present invention provide modified release of the active agent in-vitro for up to about 24 hours. In one embodiment, the compositions of the present invention provide modified release of the active agent in-vivo for up to about 24 hours. In another embodiment the compositions of the present invention provide modified release of the active agent in-vitro for up to about 12 hours. In a further embodiment, the compositions of the present invention provide modified release of the active agent in-vivo for up to about 12 hours. In a further embodiment, the compositions of the present invention provide therapeutic effect for up to about 24 hours. In another embodiment, the compositions of the present invention provide therapeutic effect for up to about 12 hours. In another embodiment, the formulations of the present invention provide modified release of the active agent in-vitro for at least about 6 hours. In a further embodiment, the formulations of the present invention provide modified release of the active agent in-vitro for at least about 8 hours. In a further embodiment is provided use of the modified release compositions of the present invention for the prevention, treatment, management or mitigation of various disease conditions or disorders depending on the active agent employed.

In another embodiment, the invention also relates to modified release compositions comprising at least one second active agent in addition to at least one active agent present in the compositions. In one embodiment, the second active agent is for immediate release. In another embodiment, the second active agent is for modified release. In another embodiment, the second active agent is different than the first active agent that is delivered in a modified manner. In still another embodiment the second active agent is complexed with ion-exchange resin. In another embodiment the second active agent is not complexed with ion exchange resin. Such a second active agent includes, but is not limited to, the list of active agents discussed above under active agents.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed, but that the invention will include all embodiments falling within the scope thereof. Details of the present invention, including its objects and advantages, are provided in the non-limiting exemplary illustrations below.

### EXAMPLES

### Example 1

### Modified Release Tolterodine Tartrate Suspension (4 mg/5 ml)

(a) Preparation of Modified Release Beads

TABLE 1
Composition of solvated bead matrix

| Ingredients | mg |
|---|---|
| Tolterodine tartrate | 4.0 |
| Sodium polystyrene sulfonate, USP | 8.0 |
| Sucrose polystearate (HLB 1) | 8.0 |
| Polyethylene glycol, USPNF | 4.0 |

Procedure: Tolterodine tartrate was complexed with the ion exchange resin sodium polystyrene sulfonate in water under stirring. The drug-resin complex formed was filtered, dried and blended with sucrose polystearate. This blend was further solvated with polyethylene glycol.

TABLE 2
Composition of release modifier coating

| Ingredients | % w/w |
|---|---|
| Ethyl cellulose, USP | 0.42 |
| Dibutyl sebacate, USPNF | 0.12 |
| Isopropyl alcohol | 5.62 |
| Water | q.s. |

Procedure: The solvated bead matrix containing tolterodine tartrate-resin complex and sucrose polystearate was coated with release modifier coating of table 2 to a level of 20% weight gain.

(b) Preparation of Modified Release Suspension

TABLE 3
Composition of suspension base

| Ingredients | % w/w |
|---|---|
| Pharma grade sugar | 35.0 |
| Methylparaben, USPNF | 0.1 |
| Propylparaben, USPNF | 0.01 |
| Disodium EDTA, USPNF | 0.005 |
| Guar gum, USPNF | 0.80 |
| Xanthan gum, USPNF | 0.20 |
| Sodium carboxymethyl cellulose, USPNF | 0.30 |
| High fructose corn syrup, USPNF | 12.0 |
| FD & C Yellow | 0.02 |
| Natural orange flavor | 0.18 |
| Polyoxyethylene sorbitan fatty acid ester, USPNF | 0.1 |
| Citric acid, USPNF | 0.25 |
| Purified water | q.s to 100 |

Procedure: Methylparaben, propylparaben, disodium EDTA were dissolved in purified water at 85-90° C. Pharma grade sugar was added to above solution under stirring and solution was subsequently cooled to room temperature. Guar gum, xanthan gum and sodium carboxymethyl cellulose were dispersed in high fructose corn syrup under stirring. This dispersion was added to the above sugar syrup under stirring to get uniform dispersion. Solution of color and flavor were then added. The tolterodine-resin complex and sucrose polystearate based modified release beads prepared above were treated with polyoxyethylene sorbitan fatty acid ester and then added to the above dispersion. Citric acid was then added and final volume was adjusted with purified water. This tolterodine suspension was palatable and has a desired modified release profile.

### Example 2

### Modified Release Oxybutynin Hydrochloride Suspension (10 mg/5 ml)

(a) Preparation of Modified Release Beads

TABLE 4
Composition of solvated bead matrix

| Ingredients | mg |
|---|---|
| Oxybutynin HCl | 10 |
| Sodium polystyrene sulfonate, USP | 10 |
| Sucrose distearate (HLB 6) | 10 |
| Polyethylene glycol, USPNF | 5 |

Procedure: Oxybutynin hydrochloride was complexed with the ion exchange resin—sodium polystyrene sulfonate in water under stirring. The drug-resin complex formed was filtered, dried and blended with sucrose distearate. This blend was further solvated with polyethylene glycol.

TABLE 5
Composition of release modifier coating

| Ingredients | % w/w |
|---|---|
| Ethyl cellulose, USP | 0.45 |
| Acetyl triethyl citrate, USPNF | 0.16 |
| Isopropyl alcohol | 8.43 |
| Water | q.s. |

Procedure: The solvated bead matrix containing oxybutynin hydrochloride-resin complex and sucrose distearate was coated with release modifier coating of table 5 to a level of 20% weight gain.

(b) Preparation of Modified Release Suspension

TABLE 6
Composition of suspension base

| Ingredients | % w/w |
|---|---|
| Pharma grade sugar | 35.0 |
| Methylparaben, USPNF | 0.1 |
| Propylparaben, USPNF | 0.01 |
| Disodium EDTA, USPNF | 0.005 |
| Guar gum, USPNF | 0.80 |
| Xanthan gum, USP NF | 0.40 |
| Sodium carboxymethyl cellulose, USPNF | 0.20 |
| High fructose corn syrup, USPNF | 12.0 |
| FD & C Yellow | 0.02 |
| Natural orange flavor | 0.18 |
| Polyoxyethylene sorbitan fatty acid esters, USPNF | 0.1 |
| Citric acid, USPNF | 0.25 |
| Purified water | q.s to 100 |

Procedure: Methylparaben, propylparaben, disodium EDTA were dissolved in purified water at 85-90° C. Pharma grade sugar was added to above solution under stirring and solution was subsequently cooled to room temperature. Guar gum, xanthan gum and sodium carboxymethyl cellulose were dispersed in high fructose corn syrup under stirring. This dispersion was added to the above sugar syrup under stirring to get uniform dispersion. Solution of color and flavor were then added. The oxybutynin-resin complex and sucrose distearate based modified release beads prepared above were treated with polyoxyethylene sorbitan fatty acid esters and then added to the above dispersion. Citric acid was then added and final volume was adjusted with purified water. This oxybutynin suspension was palatable and has a desired modified release profile.

### Example 3

### Modified Release Ziprasidone Hydrochloride Suspension (10 mg/5 ml)

(a) Preparation of Modified Release Beads

TABLE 7
Composition of solvated bead matrix

| Ingredients | mg |
|---|---|
| Ziprasidone hydrochloride | 10 |
| Sodium polystyrene sulfonate, USP | 10 |
| Sucrose palmitate (HLB 16) | 10 |
| Polyethylene glycol, USPNF | 4 |

Procedure: Ziprasidone hydrochloride was complexed with the ion exchange resin—sodium polystyrene sulphonate in water under stirring. The drug-resin complex formed was filtered, dried and blended with sucrose palmitate. This blend was further solvated with polyethylene glycol.

TABLE 8
Composition of release modifier coating

| Ingredients | % w/w |
|---|---|
| Ethyl cellulose, USP | 0.63 |
| Triethyl citrate, USPNF | 0.19 |
| Isopropyl alcohol | 8.43 |
| Water | q.s. |

Procedure: The solvated bead matrix containing ziprasidone hydrochloride-resin complex and sucrose palmitate was coated with the release modifier coating of table 8 to a level of 20% weight gain.

(b) Preparation of Modified Release Suspension

TABLE 9
Composition of suspension base

| Ingredients | % w/w |
|---|---|
| Pharma grade sugar | 35.0 |
| Methylparaben, USPNF | 0.1 |
| Propylparaben, USPNF | 0.01 |
| Disodium EDTA, USPNF | 0.005 |
| Xanthan gum, USPNF | 0.10 |
| Sodium carboxymethylcellulose, USPNF | 0.3 |
| Guar gum, USPNF | 0.6 |
| High fructose corn syrup, USPNF | 12.0 |
| F D&C Yellow | 0.02 |
| Natural orange flavor | 0.18 |
| Polyoxyethylene sorbitan fatty acid esters, USPNF | 0.1 |
| Citric acid, USPNF | 0.25 |
| Purified water | q.s to 100 |

Procedure: Methylparaben, propylparaben, disodium EDTA were dissolved in purified water at 85-90° C. Pharma grade sugar was added to above solution under stirring and solution was subsequently cooled to room temperature. Xanthan gum was dispersed in high fructose corn syrup under stirring. This dispersion was added to the above suspension base under stirring to get uniform dispersion. Solution of color and flavor was then added. The ziprasidone-resin complex and sucrose palmitate based modified release beads prepared above were treated with polyoxyethylene sorbitan fatty acid esters and then added to the above dispersion. Citric acid was then added and final volume was adjusted with purified water. This ziprasidone suspension was palatable and has a desired modified release profile.

1. A modified release composition comprising multitude of modified release beads comprising a matrix of drug-ion exchange resin complex and at least one non-polymeric multifunctional excipient; substantially coated with at least one outer release rate modifying layer, wherein the non-polymeric multifunctional excipient is a fatty acid ester, wax, fatty acid, fatty alcohol, hydrogenated vegetable oil or any combination thereof.

2. The composition of claim 1 wherein the drug-ion exchange resin complex comprises at least one active agent and at least one ion exchange resin.

3. The composition of claim 2 wherein the active agent is psychostimulant, antihistamine, expectorant or mucolytic, anti-tussive agent, serotonin and norepinephrine reuptake inhibitor, sympatholytic, antimuscarinic and urinary antispasmodic, PDE-5 inhibitor, anti-Alzheimer's agent, analgesic, decongestant, analeptic agent; anesthetic agent; anti-asthmatic, anti-arthritic agent; anti-cancer agent; anti-cholinergic agent; anti-convulsant agent, anti-depressant agent; antidiabetic; anti-helminthic agent; anti-diarrheal agent; anti-epileptic, anti-hyperlipidemic agent; antihypertensive, antihypotensive, peripheral vasodilators or vasoconstrictors, respiratory agents, anti-infective agent; anti-inflammatory agent; non-steroidal anti-inflammatory agent, anti-emetic, anti-migraine agent; anti-neoplastic agent; anti-tubercular agent; antibiotic, antacid; antiulcer agent; anti-Parkinsonism drug; anti-pruritic agent; antipsychotic agent; anti-pyretic agent; anti-spasmodic, anti-viral agent; appetite suppressant; attention deficit hyperactivity disorder treating agent; cardiovascular agent, calcium channel blocker, antianginal agent; central nervous system agent; beta-blocker; antiarrhythmic agent; bronchodilator, central nervous system stimulant; diuretic, genetic material; hormonolytic; hypnotic; hypercalcemic; hypoglycemic agent; immunosuppressive agent; beta-agonist; narcotic antagonist; nicotine; nutritional agent; parasympatholytic; peptide drug; antihemorrhoidal; psychostimulant; psychotropic; mucolytic; sedative; laxative; vitamin; sialagogue, steroid; sympathomimetic; tranquilizer; vasodilator, antianxiety drug, antidepressant, antipyretic analgesic, hypnotic, drug for the treatment of respiratory system disorders, coronary dilator, calcium antagonist, chemotherapeutic drug, antiprotozoan drug and mixtures thereof.

4. The composition of claim 2 wherein the active agent is amphetamine, amphetaminil, atomoxetine, dexmethylphenidate, dextroamphetamine, dextromethamphetamine, fencamfamine, fenethylline, lisdexamfetamine, methylphenidate, mesocarb, pemoline, pipradrol, prolintane, dimenhydrinate, diphenhydramine, chlorpheniramine, brompheniramine, dexchlorpheniramine, hydroxyzine, dexbrompheniramine, fexofenadine, terfenadine, cetirizine, levocetirizine, fexofenadine hydrochloride or dl-chlorpheniramine maleate, ambroxol, bromhexine, carbocisteine, domiodol, guaifenesin, codeine, dextromethorphan, hydrocodone, dihydrocodeine phosphate, codeine phosphate, no scapine hydrochloride, phenylpropanolamine hydrochloride, potassium guaiacolsulfonate, cloperastine fendizoate, levocloperastine fendizoate, dextromethorphan hydrobromide, cloperastine hydrochloride, clovoxamine, desvenlafaxine, duloxetine, levomilnacipran, eclanamine, milnacipran, sibutramine, venlafaxine, alaproclate, citalopram, escitalopram, femoxetine, fluoxetine, fluvoxamine, indalpine, ifoxetine litoxetine, omiloxetine, panuramine, paroxetine, pirandamine, seproxetine, sertraline zimelidine, clonidine, guanfacine, methyldopa, iloperidone, ocaperidone, paliperidone, risperidone, lurasidone, perospirone, revospirone, tiospirone, ziprasidone, chlorpromazine hydrochloride, chlorprothexene hydrochloride, darifenacin, emepronium, fesoterodine, flavoxate, imidafenacin, meladrazine, mirabegron, oxybutynin, propiverine, solifenacin, terodiline, tolterodine, trospium chloride, acetildenafil, aildenafil, avanafil, icariin, lodenafil, mirodenafil, nitrosoprodenafil, sildenafil, sulfoaildenafil, tadalafil, udenafil, vardenafil, memantine, neramexane (1, 3, 3, 5, 5-pentamethylcyclohexan-1-amine), donepezil, tacrine, rivastigmine, galantamine, physostigmine, neostigmine, Huperzine A, icopezil (CP-118954, 5,7-dihydro-3-[2-[1-(phenylmethyl)-4-piperidinyl]ethyl]-6H-pyrrolo-[4,5-1]-1,2-benzisoxazol-6-one maleate), ER-127528 (4-[(5,6-dimethoxy-2-fluoro-1-indanon)-2-yl]methyl-1-(3-fluorobenzyl) piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), metrifonate (T-588; (−)-R-α-[[2-(dimethylamino)ethoxy]methyl]benzo[b] thiophene-5-methanol hydrochloride), FK-960 (N-(4-acetyl-1-piperazinyl)-p-fluorobenzamide-hydrate), TCH-346 (N-methyl-N-2-pyropinyldibenz[b,f]oxepine-10-methanamine), SDZ-220-581 ((S)-α-amino-5-(phosphonomethyl)-[1,1′-biphenyl]-3-propionic acid), tarenflurbil, tramiprosate, clioquinol, aspirin, morphine, dihydromorphine, oxycodone, selegiline, rasagiline, entacapone, tolcapone, alfentanil, allyprodine, alphaprodine, anileridne, benzylmorphine, bezitramide, buprenorphine, butorphanol, clonitazene, cyclazocine, desmorphine, dextromoramide, dexocine, diampromide, dimexoxadol, dimepheptanol, dimethylthiambutene, dioxaphetly butyrate, dipipanone, eptazocine, ethotheptazine, ethylmethylthiambutene, ethylmorphine, etonitazene fentanyl, hydromorphone, hydroxpethidine, isomethadone, ketobermidone, levallorphan, levorphanol, levophenacylmorphan, lofentanil, meperidine, meptazinol metazocine, methadone, metopon, morphine sulfate, myrophine, nalbuphine, narceine, cicomorphine, norlevorphanol, nomethadonel nalorphine, normophine, norpipanone, ixmymorphone, papavretum, pentazocine, phenadoxone, phenmorphan, phenazocine, phenoperidine, iminodine, piritamide, propheptazine, promedol, properidine, propiram, proposyphene, sufenanil, tramadol, tiline, phenylephrine, pseudoephedrine, theophylline, phenobarbital sodium, phenytoin sodium, valproate sodium barbiturates, amylobarbitone sodium, butabarbital sodium, secobarbital sodium, phenytoin, meprobamate, nitrezepam, captopril, propranolol, hydralazine hydrochloride, propranolol hydrochloride, clonidine hydrochloride, tolazoline hydrochloride, predinisolone, prednisolone sodium phosphate, albuterol, albuterol sulfate, terbutaline, naproxen, diclofenac, indomethacin, ibuprofen, sulindac, meclofenamate sodium, tolmetin sodium, metoclopramide, oleandomycin phosphate, tetracycline hydrochloride, fradiomycin, sulfate, amoxicillin, dicloxacillin sodium, pivmecillinam hydrochloride, carbenicillin indanyl sodium, atropine, scopolamine, scopolamine hydrobromide, metixene hydrochloride, dicyclomine hydrochloride, dl-methylephedrine hydrochloride, dl-methylephedrine saccharinate; ethacrynic acid, bendrofluazide, nifedipine, papaverine, diltiazem, nicardipine, chlordiazepoxide hydrochloride, diazepam, alprazolam, imipramine hydrochloride, riseridone, sertraline hydrochloride, paroxitene hydrochloride, venlafaxine hydrochloride, sodium salicylate, etafenone hydrochloride, verapamil hydrochloride, sulfisomidine sodium, kanamycin sulfate, amodiaquine hydrochloride, dl-methyl-ephedrine hydrochloride, dehydrocholic acid, diflunisal, fenoprofen, furosemide, gemfibrozil, progencid, sulindac, salicylic acid, acetylsalicylic acid, acetophenazine, amitriptyline, benztropine, biperiden, bromodiphenhydramine, carbinoxamine, chloperastine, chlorcyclizine, chorpheniramine, chlorphenoxamine, chlorpromazine, clemastine, clomiphene, cyclizine, cyclobenzaprine, cyproheptadine, desipramine, dicyclomine, diphemanil, doxepin, doxylamine, ergotamine, fluphenazine, haloperidol, hydroxychloroquine, hyoscyamine, levopropoxyphene, maprotiline, meclizine, mepenzolate, meperidine, mephentermine, mesoridazine, metformin, methylepherdine, methdilazine, methscopolamine, methysergide, metoprolol, nortriptylene, noscapine, nylindrin, oxymorphone, orphenadrine, phendimetrazine, phentermine, phenylpropanolamine, pyrilamine, tripelennamine, triprolidine, promazine, propoxyphene, propanolol, quinidine, aminocaproic acid, aminosalicylic acid, isoxurprine, melphalan, nalidixic acid, paraaminosaliclic acid, chloropheniramine, niacin, methylphenidate hydrochloride, dexmethylphenidate hydrochloride, oxymorphone hydrochloride, hydrocodone bitartrate, albuterol sulfate, albuterol phosphate, chlorpheniramine maleate, metformin hydrochloride, oxybutynin hydrochloride, saligenine hydrochloride, cetrizine hydrochloride, ranitidine hydrochloride or combinations thereof in the form of free base or acid or pharmaceutically acceptable salts, prodrugs, active metabolites, polymorphs, solvates, hydrates, enantiomers, optical isomers, tautomers or racemic mixtures thereof.

5. The composition of claim 2 wherein the ion exchange resin is a cation exchange resin or an anion exchange resin or any combination thereof; and the cation exchange resin is selected from a copolymer of methacrylic acid and divinylbenzene, a sodium polystyrene sulfonate resin, a sulfonated copolymer of styrene and divinylbenzene, a crosslinked polyacrylic acid resin, a polyacrylate resin, a crosslinked carboxylic acid resin, a crosslinked sulfonic acid resin, a crosslinked phosphonic acid resin, zeolite or any combination thereof.

6-7. (canceled)

8. The composition of claim 1 wherein the fatty acid ester is sucrose fatty acid ester, glyceryl fatty acid ester, fatty acid alkyl ester, propylene glycol ester of fatty acid, or any combination thereof.

9. The composition of claim 8 wherein the sucrose fatty acid ester is sucrose stearate, sucrose distearate, sucrose polystearate, sucrose palmitate, sucrose monopalmitate, sucrose dipalmitate, sucrose oleate, sucrose monooleate, sucrose dioleate, sucrose laurate, sucrose monolaurate, sucrose dilaurate, sucrose behenate, sucrose erucate, sucrose caprate, sucrose monocaprate, sucrose dicaprate, sucrose caprylate, sucrose monocaprylate, sucrose dicaprylate, sucrose myristate, sucrose monomyristate, sucrose dimyristate, sucrose linolenate, sucrose monolinolenate, sucrose dilinolenate, sucrose ester of mixed fatty acids, sucrose oligoester, or any mixture thereof.

10. The composition of claim 8 wherein the glyceryl fatty acid esters are mono-, di-and triglycerides, glyceryl behenate; glyceryl monostearate, glyceryl tripalmitate, glyceryl monopalmitate, glyceryl distearate, glyceryl tristearate glyceryl palmitostearate, glyceryl dipalmitate, glyceryl monoleate, glyceryl monolaurate, glyceryl dilaurate, glyceryl trilaurate, glyceryl didocosanoate glycrl tridocosanoate, glyceryl monodocosanoate, glyceryl monocaprote, glyceryl dicaproate, glyceryl tricaproate, glyceryl monomyristate, giyberyl dimyistate, glyceryl trimyristate, glyceryl monodecenoate, glyceryl didecenoate, glycery! tridecenoate; glycowax-932; lauroyl macrogol-32 glycerides; stearoyl macrogol-32 glyceride; fatty acid esters such as those having a fatty acid chain length of about C10-C.40; glyceryl monoarachidonate, glyceryl monolinoleate, glyceryl monolinolenate, glyceryl monopalmitoleate, glyceryl monocaprylate, glyceryl monococoate, glyceryl monocollagenate, glyceryl monoerucate, glyceryl monohydroxystearate, glyceryl monoisopalmitate, glyceryl monolinoleate, glyceryl monolinolenate, glyceryl monomyristate, glyceryl monopentadecanoate, glyceryl monopolyacrylate, glyceryl monotallowate, glyceryl monothiopropionate, glyceryl monocundecylenate or any mixtures thereof.

11. The composition of claim 8 wherein the fatty acid alkyl esters are isopropyl monoarachidonate, isopropyl monolaurate, isopropyl monolinoleate, isopropyl monolinolenate, isopropyl monomyristate, isopropyl monopalmitoleate, isopropyl monooleate, and isopropyl monostearate; methyl monoarachidonate, methyl monolaurate, methyl monolinoleate, methyl monolinolenate, methyl monomyristate, methyl monopalmitoleate, methyl monooleate, methyl monostearate or any mixtures thereof.

12. The composition of claim 8 wherein the propylene glycol esters of fatty acid are propylene glycol monoarachidonate, propylene glycol monolaurate, propylene glycol monolinoleate, propylene glycol monolinolenate, propylene glycol monomyristate, propylene glycol monopalmitoleate, propylene glycol monooleate, propylene glycol monostearate or any combination thereof.

13. The composition of claim 1 wherein the non-polymeric multifunctional excipient is sucrose fatty acid ester or mixtures thereof.

14. The composition of claim 1 wherein the coating comprises at least one release modifier, said release modifier being a water-insoluble release modifier, a water-soluble release modifier or any combination thereof; and the water-insoluble release modifier is selected from a polymeric water-insoluble release modifier, a non-polymeric water-insoluble release modifier or any combination thereof.

15. (canceled)

16. The composition of claim 14 wherein the polymeric water-insoluble release modifier is polyvinyl acetate, polyvinyl chloride, polyvinyl carbonate, ethyl cellulose, nitrocellulose, vinylidene chloride-acrylonitrile copolymer, acrylonitrile-styrene copolymer, ethylene vinyl acetate, cellulose acetate, cellulose acetate phthalate, cellulose acetate butyrate, copolymer of vinyl pyrrolidone, hydroxypropylmethylcellulose phthalate, methacrylic acid copolymer, or methacrylate copolymer or any combination thereof; the non-polymeric water insoluble release modifier is selected from a fatty acid, long chain alcohol, fat, oil, wax, phospholipid, eicosnoid, terpene, steroid, resin or any combination thereof.

17. (canceled)

18. The composition of claim 14 wherein the water-soluble release modifier is polyvinylpyrrolidone, poloxamer, guar gum, xanthan gum, gum arabic, tragacanthan, cellulose derivatives such as hydroxypropylmethylcellulose, hydroxypropyl cellulose, methylcellulose, and hydroxyethyl cellulose, carboxymethylethyl cellulose, hydroxyethylmethyl carboxymethyl cellulose, hydroxyethyl methyl cellulose, carboxymethyl cellulose, methylhydroxyethyl cellulose, or methylhydroxypropyl cellulose or any combination thereof.

19. The composition of claim 1 wherein the pharmaceutically acceptable excipient is a binder, disintegrant, superdisintegrant, diluent, salivating agent, surfactant, flavor, sweetener, colorant, souring agent, solvating agent, viscolizer, glidant, lubricant, solubilizer, stabilizer, suspending agent, preservative, cosolvent, anti-caking agent, or buffer or any combination thereof.

20. The composition of claim 1 wherein the formulation is in the form of a liquid, a solid or a semisolid preparation; said liquid preparation being suspension and said solid preparation being capsule, tablet, caplet, orally disintegrating tablet, dispersible tablet, dry suspension for reconstitution, granule, wafer or bite-dispersion tablet.

21. The composition of claim 1 wherein the formulation is in the form of a suspension.

22. The composition of claim 1 wherein the modified release composition further comprises an additional active agent.

23. (canceled)

24. A modified release suspension comprising multitude of modified release beads comprising a matrix of drug-ion exchange resin complex and at least one non-polymeric multifunctional excipient; substantially coated with at least one outer release rate modifying layer.

25. A modified release suspension comprising multitude of modified release beads comprising a matrix of drug-ion exchange resin complex and at least one sucrose fatty acid ester; substantially coated with at least one outer release rate modifying layer.
